(19) United States
(12) Patent Application Publication
(10) Pub. No.: US 2025/0035660 A1
(43) Pub. Date: Jan. 30, 2025

(54) BIOLOGICAL SAMPLE PROCESSING APPARATUS

(71) Applicant: Canon Medical Systems Corporation, Otawara-shi (JP)

(72) Inventors: Mayumi SAKAGAMI, Ibaraki (JP); Tomoki FUJITO, Nasushiobara (JP); Koji HIRATA, Kasukabe (JP); Kohei WATANABE, Utsunomiya (JP); Kanako AIZAWA, Yaita (JP)

(73) Assignee: Canon Medical Systems Corporation, Otawara-shi (JP)

(21) Appl. No.: 18/775,676

(22) Filed: Jul. 17, 2024

(30) Foreign Application Priority Data
Jul. 24, 2023 (JP) 2023-120026

(51) Int. Cl. G01N 35/10; G01N 35/00

(52) U.S. Cl. CPC G01N 35/1002; G01N 35/00732; G01N 2035/00475

(57) ABSTRACT

A biological sample processing apparatus includes a holder. The holder detachably holds a liquid supplier. The liquid supplier includes a storage and an ejector. The storage stores a liquid used for processing a biological sample. The ejector pushes the liquid out from inside the storage to eject the liquid directly to an ejection destination.

### CROSS-REFERENCE TO RELATED APPLICATIONS

This application is based upon and claims the benefit of priority from Japanese Patent Application No. 2023-120026, filed Jul. 24, 2023, the entire contents of which are incorporated herein by reference.

### FIELD

Embodiments described herein relate generally to a biological sample processing apparatus.

### BACKGROUND

A cell processing apparatus and an automated culturing apparatus serve as biological sample processing apparatuses for processing a biological sample. A conventional cell processing apparatus has a storage for storing a reagent or a cell suspension, to which a commercially available reagent bottle or a reagent bag is attached. The storage is connected, via a passage such as a tube, to a container to which a reagent or a cell suspension is supplied. Since a liquid such as a reagent is supplied via a passage in such a biological sample processing apparatus, a residue of a reagent or the like is attached to an inner wall of the passage, leading to the risk of contamination.

### BRIEF DESCRIPTION OF THE DRAWINGS

FIG. 1 is a diagram showing a configuration of a biological sample processing apparatus according to a first embodiment.

FIG. 2 is a diagram showing an example of a configuration of a blood cell separation mechanism according to the first embodiment.

FIG. 3 is a diagram showing an example of a configuration of a liquid supplier according to the first embodiment.

FIG. 4 is a diagram showing an example of a configuration of a liquid supplier according to the first embodiment.

FIG. 5 is a diagram showing an example of a configuration of a liquid supplier according to the first embodiment.

FIG. 6 is a flowchart illustrating a procedure of a blood cell separation process according to the first embodiment.

FIG. 7 is a flowchart illustrating a procedure of blood cell separation performed by the biological sample processing apparatus according to the first embodiment.

FIG. 8 is a flowchart illustrating a waste disposal procedure performed by the biological sample processing apparatus according to the first embodiment.

FIG. 9 is a diagram showing a state of the blood cell separation mechanism when the blood cell separation is started.

FIG. 10 is a diagram showing an operation of the blood cell separation mechanism performed in step S202 in FIG. 7.

FIG. 11 is a diagram showing an operation of the blood cell separation mechanism performed in step S203 in FIG. 7.

FIG. 12 is a diagram showing an operation of the blood cell separation mechanism performed in step S204 in FIG. 7.

FIG. 13 is a diagram showing an operation of the blood cell separation mechanism performed in step S206 in FIG. 7.

FIG. 14 is a diagram showing an operation of the blood cell separation mechanism performed in step S105 in FIG. 6.

FIG. 15 is a diagram showing a configuration of a biological sample processing apparatus according to a second embodiment.

FIG. 16 is a diagram showing an example of a configuration of a factor introduction mechanism according to the second embodiment.

FIG. 17 is a flowchart illustrating a procedure of a factor introduction process according to the second embodiment.

FIG. 18 is a flowchart illustrating a procedure of factor introduction performed by the biological sample processing apparatus according to the second embodiment.

FIG. 19 is a diagram showing a state of the factor introduction mechanism when the factor introduction is started.

FIG. 20 is a diagram showing an operation of the factor introduction mechanism performed in step S502 in FIG. 18.

FIG. 21 is a diagram showing an operation of the factor introduction mechanism performed in step S503 in FIG. 18.

FIG. 22 is a diagram showing an operation of the factor introduction mechanism performed in step S504 in FIG. 18.

FIG. 23 is a diagram showing an operation of the factor introduction mechanism performed in step S505 in FIG. 18.

FIG. 24 is a diagram showing an example of a configuration of a liquid supplier according to a first modification.

FIG. 25 is a diagram showing an example of a configuration of a liquid supplier according to a second modification.

### DETAILED DESCRIPTION

In general, according to an embodiment, a biological sample processing apparatus includes a holder. The holder detachably holds a liquid supplier. The liquid supplier includes a storage and an ejector. The storage stores a liquid used for processing a biological sample. The ejector pushes the liquid out from inside the storage to eject the liquid directly to an ejection destination.

Hereinafter, embodiments of a biological sample processing apparatus will be described in detail with reference to the accompanying drawings. The biological sample processing apparatus is an apparatus that automatically processes biological samples such as blood, a peripheral blood mononuclear cell (hereinafter referred to as a “PBMC”) and the like. Processing a biological sample can be taken to mean, for example, the processing of a cell for making an induced pluripotent stem cell (hereinafter referred to as an “iPS cell”). For example, the processing of a biological sample is to separate a blood cell in blood or introduce a factor into a PBMC. The biological sample processing apparatus has a cartridge-type liquid supplier that stores a liquid used to process a biological sample and is capable of directly ejecting the stored liquid such as a reagent. The liquid supplier is attached in a detachable fashion. In the description below, constituents having substantially the same functions and configurations will be denoted by the same reference symbols, and a repeat description of such constituents will be given only where necessary.

### First Embodiment

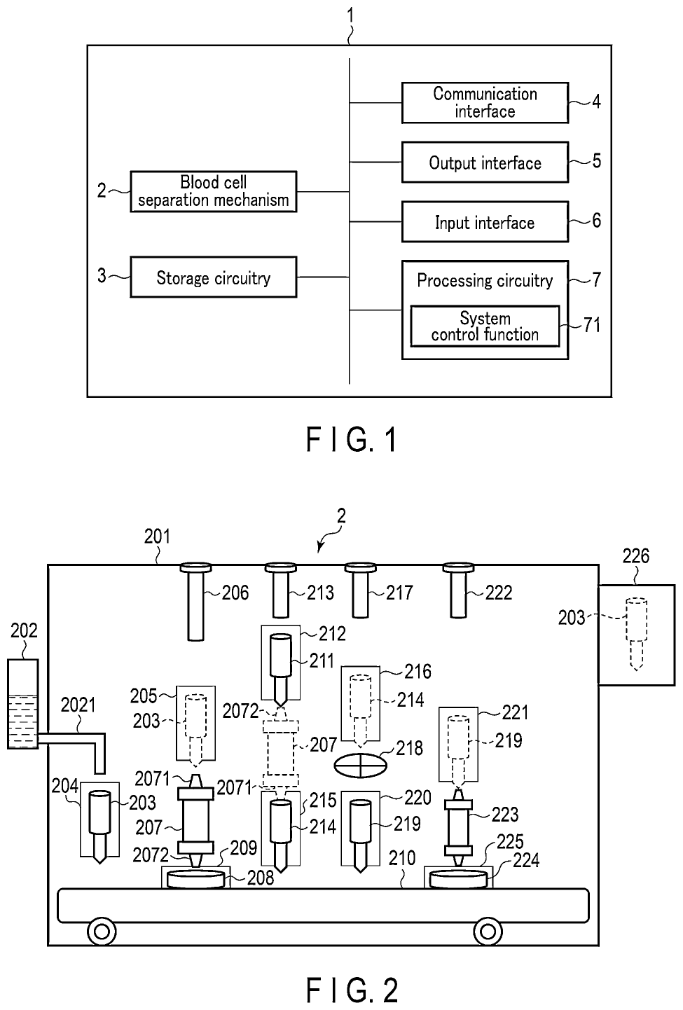

FIG. 1 is a block diagram showing an example of a configuration of a biological sample processing apparatus 1 according to a first embodiment. The biological sample processing apparatus 1 of the present embodiment is a blood cell separating apparatus that automatically separates, from blood, a blood cell necessary for making an iPS cell. As shown in FIG. 1, the biological sample processing apparatus 1 includes a blood cell separation mechanism (separator) 2, an input interface 6, an output interface 5, storage circuitry 3, and control circuitry 7. The control circuitry may be rephrased as processing circuitry.

The blood cell separation mechanism 2 separates, from blood, a blood cell necessary for making an iPS cell. A detailed configuration of the blood cell separation mechanism 2 will be described later.

The storage circuitry 3 stores programs to be executed by the control circuitry 7, various types of data to be used in the processing performed by the control circuitry 7, and the like. Such programs include, for example, a program that is pre-installed in a computer via a network or a non-transitory computer-readable storage medium to cause the computer to implement each function of the control circuitry 7. The various types of data as used herein are typically forms of digital data. The storage circuitry 3 is an example of a storage.

The communication interface 4 is an interface that performs data communication with an external device via network.

The output interface 5 is connected to the control circuitry 7 and outputs a signal supplied from the control circuitry 7. The output interface 5 is realized, for example, by display circuitry, print circuitry, a voice device, and the like. The display circuitry includes, for example, a CRT display, a liquid crystal display, an organic EL display, an LED display, a plasma display, and the like. The display circuitry may also be processing circuitry that converts data showing a display target into a video signal which is then output to an external device. The print circuitry includes, for example, a printer and the like. The print circuitry may also be output circuitry that outputs data showing a print target to an external device. The voice device includes, for example, a speaker and the like. The voice device may be output circuitry that outputs a voice signal to an external device.

The input interface 6 receives, for example, various operations from an operator. The input interface 6 is realized by, for example, a mouse, a keyboard, a touch pad which allows input of instructions through a touch on its operation screen, and the like. The input interface 6 is connected to the control circuitry 7, thereby converting an operation command that is input by an operator into an electric signal which is then output to the control circuitry 7.

The input interface 6 is not limited to a component that has physical operational components such as a mouse and a keyboard. For example, the input interface 6 may be processing circuitry that receives an electric signal corresponding to an operation command input from an external input device provided separately from the biological sample processing apparatus 1 and outputs the electric signal to the control circuitry 7.

The control circuitry 7 is a processor that functions as the center of the biological sample processing apparatus 1. The control circuitry 7 executes a system control function 71 by executing a program read from the storage circuitry 3. With the system control function 71, the control circuitry 7 comprehensively controls each component of the biological sample processing apparatus 1. For example, in the system control function 71, the control circuitry 7 controls the blood cell separation mechanism 2 such that the blood cell separation described later is performed. The control circuitry 7 that implements the system control function 71 is an example of a controller.

The term “processor” used in the above description refers to, for example, circuitry such as a CPU (central processing unit), a GPU (graphics processing unit), an ASIC (application specific integrated circuit), a programmable logic device (such as a simple programmable logic device (SPLD), a complex programmable logic device (CPLD), and a field programmable gate array (FPGA)), etc. If the processor is, for example, a CPU, the processor implements the functions by reading and executing programs stored in the storage circuitry 3. On the other hand, if the processor is an ASIC, its functions are directly incorporated into the circuitry of the processor as logic circuitry, instead of being registered on a program being stored in the storage circuitry 3. Each processor of the present embodiment is not limited to be configured as a single piece of circuitry; multiple sets of independent circuitry may be integrated into a single processor that implements its functions. Furthermore, the plurality of components in FIG. 1 may be integrated into one processor to realize its function. The above description of the “processor” also applies to each embodiment and modification described below.

In the present embodiment, descriptions will be given on the premise that each function is implemented by a single processor; however, the embodiment is not limited thereto. For example, a plurality of independent processors may be combined to constitute control circuitry, and the respective processors may implement the respective functions by executing the programs. The system control function 71 may be referred to as “system control circuitry” or installed as individual hardware circuitry. The above descriptions of the respective functions implemented by the control circuitry 7 also apply to each of the embodiments and modifications described below. The control circuitry 7 may include a storage area for storing at least part of the data stored in the storage circuitry 3. The control circuitry 7 may be referred to as a “controller” or “processing circuitry”.

Next, a configuration of the blood cell separation mechanism 2 will be described in detail.

FIG. 2 is a diagram showing an example of a configuration of the blood cell separation mechanism 2 shown in FIG. 1. As shown in FIG. 2, the blood cell separation mechanism 2 has a housing 201, a blood bag 202, a mixing container 203, a first holder 204, a second holder 205, a first pump 206, a first filter 207, a waste container 208, a third holder 209, a conveyor 210, a reagent cartridge 211, a fourth holder 212, a second pump 213, a first collecting container 214, a fifth holder 215, a sixth holder 216, a third pump 217, a passage 218, a second collecting container 219, a seventh holder 220, an eighth holder 221, a fourth pump 222, a second filter 223, a third collecting container 224, a ninth holder 225, and an UV radiation unit 226. The blood cell separation mechanism 2 also has a drive mechanism. The drive mechanism actuates each of the above components of the blood cell separation mechanism 2 under the control of the control circuitry 7. The drive mechanism is realized, for example, by a robot arm, a pump, a gear, a belt conveyor, etc. For example, a syringe pump is used as the first pump 206, the second pump 213, the third pump 217, and the fourth pump 222. For example, a well plate, a flask, a dish, or a bag are used as the various containers such as the mixing container 203, the waste container 208, the first collecting container 214, the second collecting container 219, and the third collecting container 224.

The housing 201 is an exterior of the blood cell separation mechanism 2. The blood bag 202 is attached to the outer side of the housing 201. The configurations (the mixing container 203 to the UV radiation unit 226, etc.) other than the blood bag 202 are provided inside the housing 201. The blood bag 202 may be provided inside the housing 201.

The blood bag 202 is a container capable of storing blood, which is a biological sample. The blood bag 202 may store only an amount of blood used to perform single blood cell separation or a multiple-time amount of blood. For example, the blood bag 202 has air tightness so as to be able to suppress or prevent exposure of blood to the external air. The blood bag 202 is connected to a drive mechanism such as a pump and supplies blood to a blood passage 2021.

Blood is a cell fluid that includes a blood cell necessary for making an iPS cell. A blood cell necessary for making an iPS cell is a cell to be reprogrammed by an inducing factor and is a mononuclear cell such as a CD34 cell or a white blood cell that includes a CD34 cell.

The mixing container 203 is a container for mixing blood with phosphate buffered saline (hereinafter referred to as “PBS”) and preparing a mixed solution of blood and PBS. For example, the mixing container 203 has air tightness so as to be able to suppress or prevent exposure of the mixed solution to the external air. A one-time amount of PBS is prestored in the mixing container 203. The one-time amount is, for example, an amount of PBS used at one time to be mixed with blood.

The first holder 204 is a holding mechanism for holding the mixing container 203. The mixing container 203 is attached to the first holder 204 by an operator. In the state of being attached to the first holder 204, the mixing container 203 is connected to the blood bag 202 via the blood passage 2021. Blood is supplied from the blood bag 202 to the mixing container 203 through the blood passage 2021. The other end of the blood passage 2021 is connected to the blood bag 202. Thus, the mixing container 203 is connected to the blood bag 202 via the blood passage 2021. Blood is supplied from the blood bag 202 to the mixing container 203 through the blood passage 2021, and a mixed solution of blood and PBS is generated in the mixing container 203. The mixing container 203 is conveyed from the first holder 204 to the second holder 205 by a robot arm.

The second holder 205 is a holding mechanism for holding the mixing container 203 conveyed from the first holder 204. The second holder 205 is arranged between the first pump 206 and the first filter 207.

The first pump 206 is connected to the mixing container 203 held by the second holder 205. The first pump 206 is, for example, a syringe pump. The first pump 206 extends downward under the control of the control circuitry 7 and ejects the mixed solution stored in the mixing container 203 to the first filter 207.

The first filter 207 is used to capture a necessary blood cell contained in the mixed solution and separate the necessary blood cell from the blood. The first filter 207 removes unnecessary cells such as red blood cells and blood platelets from the blood and extracts necessary cells such as white blood cells.

The first filter 207 has an opening smaller than the particle sizes of white blood cells and larger than the particle sizes of red blood cells, blood platelets and the like. Thus, when the blood passes through the first filter 207, the first filter 207 allows unnecessary cells such as red blood cells and blood platelets to pass through and captures necessary cells such as white blood cells.

The first filter 207 has a first end 2071 and a second end 2072. The first end 2071 is positioned at one end of the first filter 207, and the second end 2072 is positioned at an end opposite to the first end 2071. The first filter 207 is arranged directly below the second holder 205 with the first end 2071 positioned on the upper side and the second end 2072 positioned on the lower side. A robot arm rotates around the first filter 207 that has captured necessary cells 180 degrees to overturn the first filter 207 upside down and moves the first filter 207 to directly below the fourth holder 212. In this state, the second end 2072 is positioned on the upper side and the first end 2071 is positioned on the lower side.

The waste container 208 is a container for collecting, as a waste liquid, the mixed solution having passed through the first filter 207. For example, the waste container 208 has air tightness so as to be able to suppress or prevent exposure of the mixed solution to the external air.

The third holder 209 is a holding mechanism for holding the waste container 208. The third holder 209 is positioned directly below the first filter 207. The third holder 209 is realized by the conveyor 210. The conveyor 210 is a belt conveyor for conveying the waste container 208 and the third collecting container 224. For example, the waste container 208 is conveyed to a waste collector (not shown).

The reagent cartridge 211 is a cartridge-type container capable of storing PBS. The reagent cartridge 211 is an example of a liquid supplier of the present embodiment. A one-time amount of PBS is prestored in the reagent cartridge 211. The one-time amount is, for example, an amount of PBS used at one time to collect the cells captured by the first filter 207. A multiple-time amount of PBS may be stored in the reagent cartridge 211. The reagent cartridge 211 may be referred to as a “collected liquid cartridge”. A detailed configuration of the reagent cartridge 211 will be described later.

The fourth holder 212 is a holding mechanism for holding the reagent cartridge 211. The fourth holder 212 is an example of a holder for holding the liquid supplier. The reagent cartridge 211 is attached to the fourth holder 212 by an operator. The fourth holder 212 is arranged directly below the second pump 213. The first filter 207 turned upside down is arranged directly below the fourth holder 212. In the fourth holder 212, the reagent cartridge 211 is directly connected to the first filter 207. The reagent cartridge 211 ejects PBS directly to the first filter 207 through the driving done by the second pump 213. The first filter 207 is an example of an ejection destination to which a liquid is ejected from the liquid supplier. A used reagent cartridge 211 is conveyed from the fourth holder 212 to the UV radiation unit 226 by a robot arm.

The second pump 213 is connected to the reagent cartridge 211 held by the fourth holder 212. The second pump 213 is, for example, a syringe pump. The second pump 213 extends downward under the control of the control circuitry 7 and ejects the PBS stored in the reagent cartridge 211 to the first filter 207. The second pump 213 is an example of a driver.

The first collecting container 214 is a container for collecting PBS including the cells collected from the first filter 207. For example, the first collecting container 214 has air tightness so as to be able to suppress or prevent exposure of the mixed solution to the external air.

The fifth holder 215 is a holding mechanism for holding the first collecting container 214. The fifth holder 215 is arranged directly below the first filter 207 turned upside down. The first collecting container 214 is conveyed from the fifth holder 215 to the sixth holder 216 by a robot arm.

The sixth holder 216 is a holding mechanism for holding the first collecting container 214 conveyed from the fifth holder 215. The sixth holder 216 is arranged between the third pump 217 and the passage 218.

The third pump 217 is connected to the first collecting container 214 held by the sixth holder 216. The third pump 217 is, for example, a syringe pump. The third pump 217 extends downward under the control of the control circuitry 7 and ejects the collected liquid stored in the first collecting container 214 to the passage 218.

The collected liquid that includes white blood cells passes through the passage 218, whereby PBMCs are generated. The passage 218 is, for example, a microchannel device for generating a PBMC. The passage 218 is arranged directly below the sixth holder 216.

The second collecting container 219 is a container for collecting the PBMCs generated in the passage 218. The second collecting container 219 is arranged directly below the passage 218. For example, the second collecting container 219 has air tightness so as to be able to suppress or prevent exposure of the mixed solution to the external air.

The seventh holder 220 is a holding mechanism for holding the second collecting container 219. The seventh holder 220 is arranged directly below the passage 218. The second collecting container 219 is conveyed from the seventh holder 220 to the eighth holder 221 by a robot arm.

The eighth holder 221 is a holding mechanism for holding the second collecting container 219 conveyed from the seventh holder 220. The eighth holder 221 is arranged between the fourth pump 222 and the second filter 223.

The fourth pump 222 is connected to the second collecting container 219 held by the eighth holder 221. The fourth pump 222 is, for example, a syringe pump. The fourth pump 222 extends downward under the control of the control circuitry 7 and ejects the PBMCs stored in the second collecting container 219 to the second filter 223.

The second filter 223 is a filter for performing predetermined filtering on PBMCs. The second filter 223 is arranged directly below the eighth holder 221.

The third collecting container 224 is a culture container containing a medium. Also, the third collecting container 224 collects PBMCs which have passed through the second filter 223. For example, the third collecting container 224 has air tightness so as to be able to suppress or prevent exposure of the mixed solution to the external air. A medium for establishing an iPS cell is prestored in the third collecting container 224. For example, cytokines or StemSpan of Veritas Corporation can be used as the medium.

The ninth holder 225 is a holding mechanism for holding the third collecting container 224. The ninth holder 225 is positioned directly below the second filter 223. The ninth holder 225 is realized by the conveyor 210. The conveyor 210 conveys the third collecting container 224 to a slot (not shown). An operator takes out the culture container containing a medium from the blood cell separation mechanism 2 by removing the third collecting container 224 from the slot, and performs the culturing process subsequent to the blood cell separation process.

The UV radiation unit 226 stores a used reagent cartridge 211 and irradiates the used reagent cartridge 211 with ultraviolet (UV). The UV radiation unit 226 is provided with an irradiator for emitting ultraviolet (UV), and a used reagent cartridge 211 is irradiated with ultraviolet for a certain period of time. Once the ultraviolet radiation is completed, the reagent cartridge 211 is removed from a discharge port (not shown) to be discharged to the outside and discarded. The UV radiation unit 226 is an example of a waste disposer for disposing of waste generated by the processing of a biological sample.

A small-sized passage or filter provided to the blood cell separation mechanism 2 may be configured to be attachable and detachable. For example, the first filter 207, the passage 218, and the second filter 223 may be arbitrarily attachable and detachable, and may be replaceable with a filter or passage having a desired function. The first filter 207, the passage 218, or the second filter 223 configured to be attachable and detachable is an example of an attachable and detachable processor used to process a biological sample.

Next, an example of the configuration of the reagent cartridge 211, which is a reagent cartridge of the present embodiment, will be described.

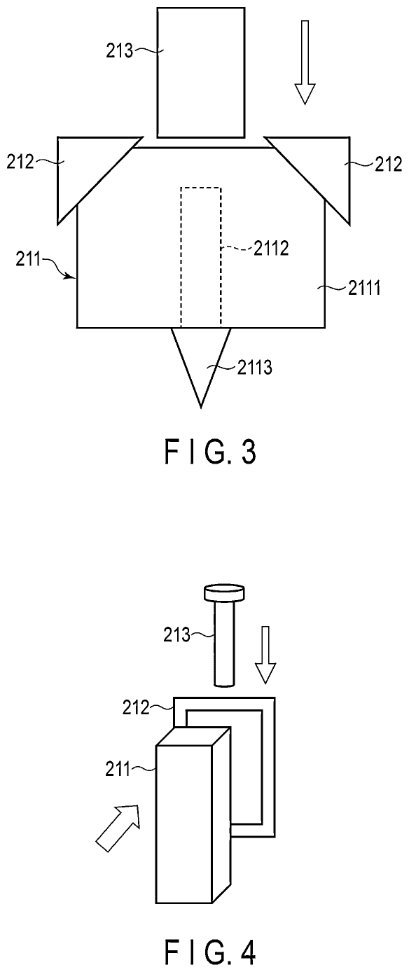

FIG. 3 is a diagram showing an example of the configuration of the reagent cartridge 211. As shown in FIG. 3, the reagent cartridge 211 has a storage 2111, a piston 2112, and an ejection unit 2113.

The storage 2111 is a reagent bag for storing PBS, which is a reagent. For example, the storage 2111 has air tightness so as to be able to suppress or prevent exposure of the PBS to the external air. The storage 2111 is an example of a storage for storing a liquid used to process a biological sample. For example, the fourth holder 212 is fixed to a predetermined position inside the housing 201. The fourth holder 212 supports the storage 2111. The exterior of the storage 2111 is formed in a shape that allows the storage 2111 to be detachably attached to the fourth holder 212. The fourth holder 212 has, for example, a fitting hole into which the storage 2111 can be fitted from a lower side or a transverse side.

The piston 2112 is provided inside the storage 2111 and attached to the second pump 213. The ejection unit 2113 is a discharge port provided in a lower part of the storage 2111. The ejection unit 2113 is a discharge port provided at a lower end of the storage 2111 and is directly connected to the first filter 207. The piston 2112 moves when driven by the second pump 213. When the piston 2112 is pushed through the driving done by the second pump 213, the PBS stored in the storage 2111 is ejected directly from the ejection unit 2113 to the first filter 207. The piston 2112 and the ejection unit 2113 are examples of an ejector for pushing a liquid out from inside the storage and ejecting the liquid directly to the ejection destination. The second pump 213 is an example of a driver for ejecting a liquid from the ejector.

The blood cell separation mechanism 2 has a mix-up preventer for preventing mix-up of the reagent cartridge 211. In the present embodiment, the fourth holder 212 is formed to allow only its corresponding liquid supplier to be attached thereto, and the storage 2111 of the reagent cartridge 211 is formed so as to be attached only to the fourth holder 212. Specifically, the fourth holder 212 is formed in a shape that allows only the reagent cartridge 211 to be attached thereto. For example, the reagent cartridge 211 is formed in a shape that allows itself to be attached to the fourth holder 212 and does not allow itself to be attached to the fitting of the blood bag 202, and the blood bag 202 is formed in a shape that allows itself to be attached to the fitting of the blood bag 202 and does not allow itself to be attached to the fourth holder 212, thereby preventing mix-ups. The fourth holder 212 and the reagent cartridge 211 thus formed are examples of a mix-up preventer.

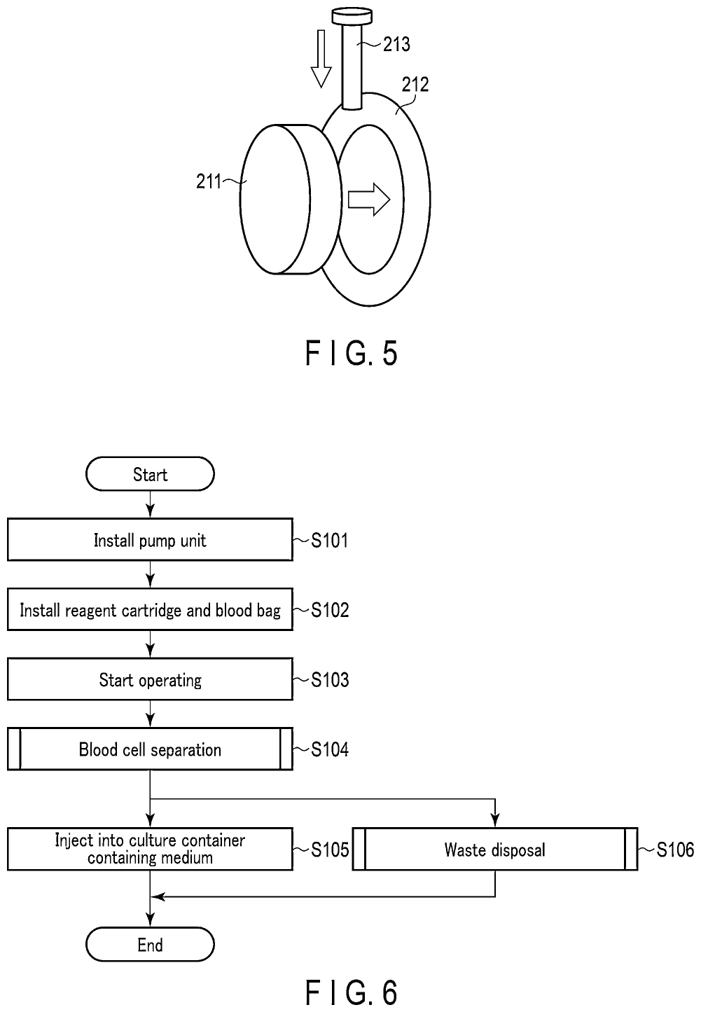

FIGS. 4 and 5 are diagrams showing an example of the reagent cartridge 211 having a mix-up prevention function. For example, the fourth holder 212 may be formed to have a rectangular fitting hole, and the contour of the reagent cartridge 211 may be formed in a rectangular shape that fits to the fitting hole, as shown in FIG. 4. Also, the fourth holder 212 may be formed to have a circular fitting hole, and the contour of the reagent cartridge 211 may be formed in a circular shape that fits into the fitting hole, as shown in FIG. 5. Also, the contour of the reagent cartridge 211 may be formed to be rectangular, and the contour of the blood bag 202 may be formed to be circular. Also, the contour of the reagent cartridge 211 may be formed to be circular, and the contour of the blood bag 202 may be formed to be rectangular.

Next, an operation of the biological sample processing apparatus 1 of the present embodiment will be described.

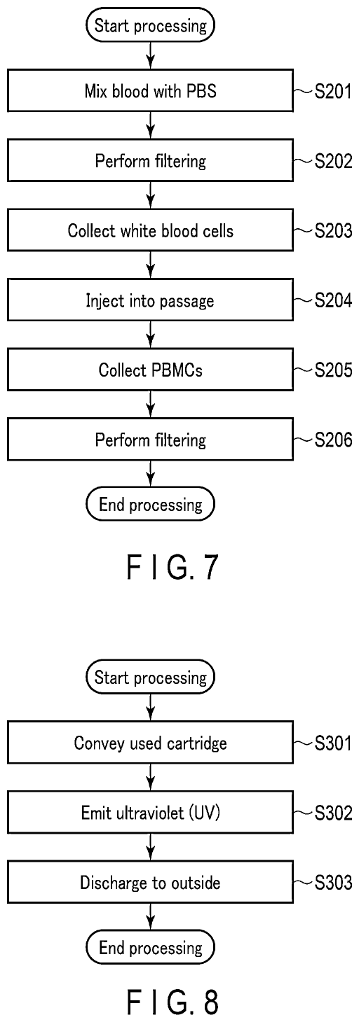

FIG. 6 is a flowchart showing an example of a procedure of a blood cell separation process performed by an operator using the biological sample processing apparatus 1. The blood cell separation process is a process of separating a blood cell necessary to make an iPS cell, such as a white blood cell, from the blood of a subject. FIG. 7 is a flowchart showing an example of a procedure of blood cell separation (described later) performed in step S104 in FIG. 6. FIG. 8 is a flowchart showing an example of a procedure of waste disposal (described later) performed in step S106 in FIG. 6. The procedure of each step described below is a mere example, and each step can be altered as appropriate to the extent possible. Omission, replacement, and addition of a step can be made as appropriate in the procedure described below according to the manner in which the embodiment will be implemented. The foregoing explanation of the procedure of each step applies to each of the embodiments and modifications described below.

### (Blood Cell Separation Process)

### (Step S101)

First, an operator installs a pump unit including the first pump 206, the second pump 213, the third pump 217, and the fourth pump 222 in a predetermined position inside the housing 201 of the blood cell separation mechanism 2. The pump unit may be referred to as a “nozzle unit”.

### (Step S102)

Next, the operator installs the blood bag 202 and the reagent cartridge 211 in the fourth holder 212. At this time, the operator can install the blood bag 202 and the reagent cartridge 211 in a correct position without mixing them up since the reagent cartridge 211 and the fourth holder 212 are provided with the above-described mix-up prevention function.

### (Step S103)

Next, the operator performs an operation to start operating the biological sample processing apparatus 1 through the input interface 6.

### (Step S104)

When the biological sample processing apparatus 1 starts to operate, the biological sample processing apparatus 1 performs the blood cell separation under the control of the control circuitry 7. In the blood cell separation, an operation of separating a blood cell from blood is automatically performed by the blood cell separation mechanism 2 under the control of the control circuitry 7.

Herein, the blood cell separation performed by the biological sample processing apparatus 1 will be explained in detail.

FIG. 7 is a flowchart showing an example of a procedure of the blood cell separation performed by the biological sample processing apparatus 1. Each processing described below is performed through the driving done by a drive mechanism such as a robot arm or the conveyor 210 under the control of the control circuitry 7, a description of which is omitted.

### (Blood Cell Separation)

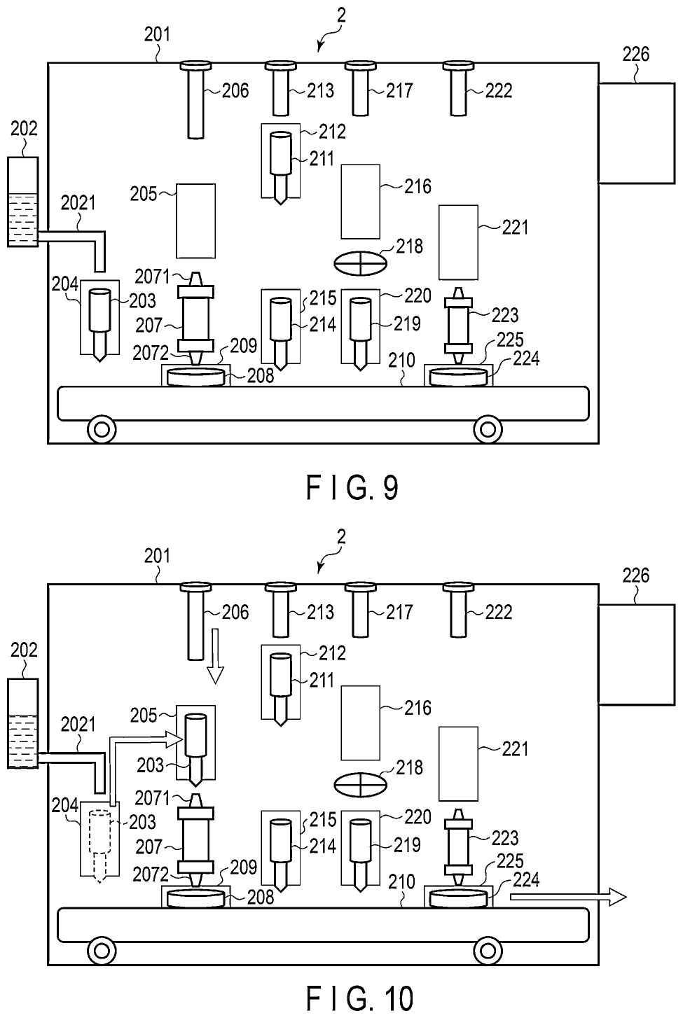

FIG. 9 is a diagram showing an internal state of the blood cell separation mechanism 2 when the blood cell separation is started. As shown in FIG. 9, to start the blood cell separation, the blood bag 202 is attached to the outer side of the housing 201, and the reagent cartridge 211 is attached to the fourth holder 212. Also, the mixing container 203 storing PBS is attached to the first holder 204. The first filter 207 is placed directly below the second holder 205, the waste container 208 is placed in the third holder 209, the first collecting container 214 is placed in the fifth holder 215, the second collecting container 219 is placed in the seventh holder 220, and the third collecting container 224 containing a medium is placed in the ninth holder 225.

### (Step S201)

First, when the blood cell separation is started, the blood cell separation mechanism 2 supplies blood from the blood bag 202 to the mixing container 203 and generates a mixed solution of the blood and PBS in the mixing container 203.

### (Step S202)

Next, the mixing container 203 moves from the first holder 204 to the second holder 205, as shown in FIG. 10. Then, through the driving done by the first pump 206, the mixed solution stored in the mixing container 203 is ejected to the first filter 207 and subjected to filtering by the first filter 207. Through the filtering, the white blood cells contained in the blood are captured by the first filter 207. The mixed solution having passed through the first filter 207 is collected, as a waste liquid, by the waste container 208 placed in the third holder 209.

### (Step S203)

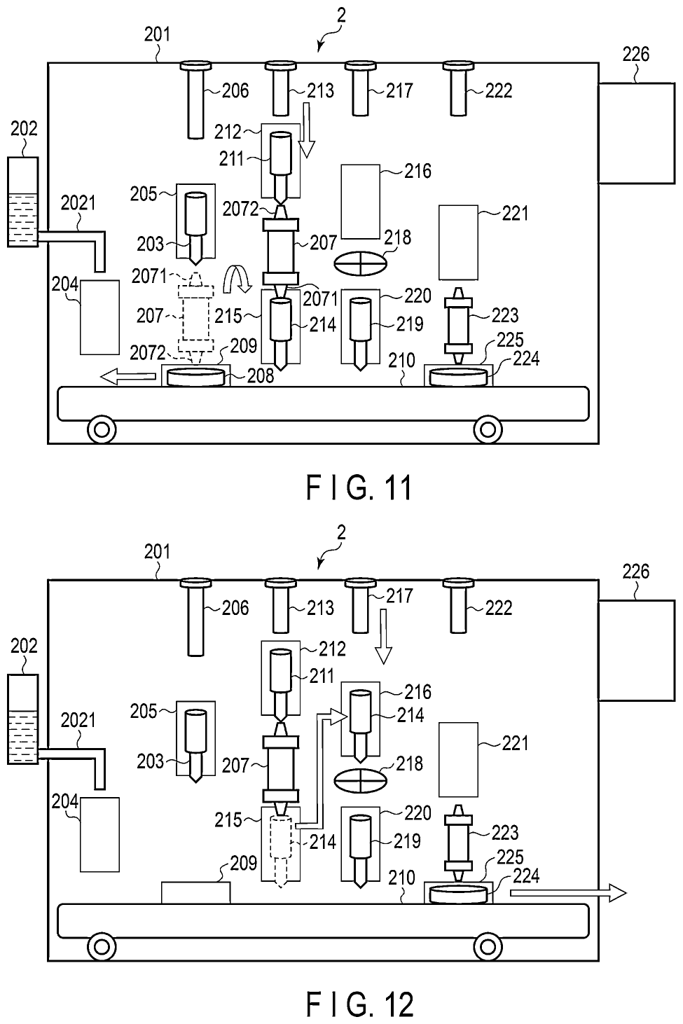

Next, the first filter 207 is rotated so as to be turned upside down and moves to a place between the fourth holder 212 and the fifth holder 215, as shown in FIG. 11. Also, the waste container 208 is conveyed to a waste collector (not shown) by the conveyor 210. Then, through the driving done by the second pump 213, a one-time amount of PBS stored in the reagent cartridge 211 is ejected to the first filter 207 turned upside down and subjected to filtering by the first filter 207. Through the filtering, the white blood cells captured by the first filter 207 are collected by the first collecting container 214 together with the PBS.

### (Step S204)

Next, the first collecting container 214 moves from the fifth holder 215 to the sixth holder 216, as shown in FIG. 12. Then, through the driving done by the third pump 217, the collected liquid stored in the first collecting container 214 is ejected to the passage 218.

### (Step S205)

Next, as a result of the collected liquid passing through the passage 218, PBMCs are generated, and the PBMCs having passed through the passage 218 are collected by the second collecting container 219.

### (Step S206)

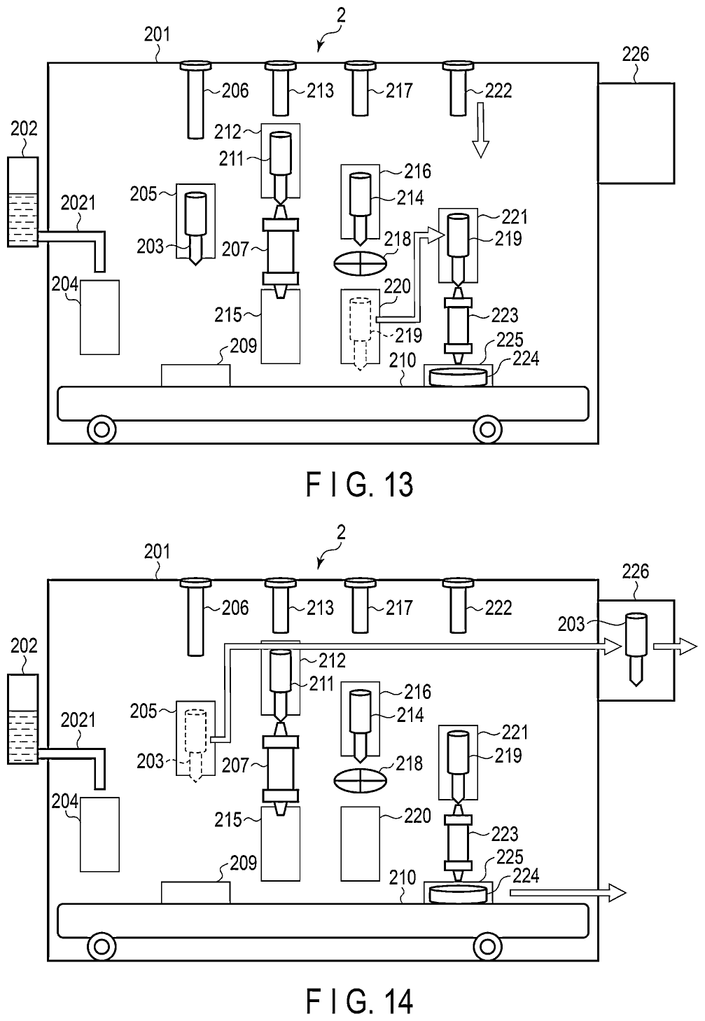

Next, the second collecting container 219 moves from the seventh holder 220 to the eighth holder 221, as shown in FIG. 13. Then, through the driving done by the fourth pump 222, the PBMCs stored in the second collecting container 219 are ejected to the second filter 223, which subjects them to filtering. The filtering process, for example, sees any step for PBMCs being performed.

In this manner, in the blood cell separation performed in step S104, PBMCs necessary to process iPS cells are obtained through a series of steps S201 through S206. When the blood cell separation is completed, step S105 and step S106 in FIG. 6 are performed simultaneously.

### (Step S105)

The operator operates the biological sample processing apparatus 1 to cause the PBMCs which have passed through the second filter 223 to be injected into the third collecting container 224 containing a medium. The operator then conveys the third collecting container 224 from the ninth holder 225 to the slot, as shown in FIG. 14, and completes the blood cell separation process.

The operator then takes out the third collecting container 224 from the biological sample processing apparatus 1 and performs a culturing process using the third collecting container 224 taken out from the biological sample processing apparatus 1.

### (Step S106)

Simultaneously with step S105, the operator performs waste disposal using the biological sample processing apparatus 1. The waste disposal is processing for sterilizing the used reagent cartridge in order to safely discard the used reagent cartridge.

FIG. 8 is a flowchart showing an example of a procedure of the waste disposal performed by the biological sample processing apparatus 1. Each processing described below is performed through the driving done by each drive mechanism of the blood cell separation mechanism 2 under the control of the control circuitry 7, a description of which is omitted.

### (Waste Disposal)

### (Step S301)

In the waste disposal, first, the used reagent cartridge 211 having ejected PBS is moved from the fourth holder 212 to the UV radiation unit 226, as shown in FIG. 14.

### (Step S302)

Next, the used reagent cartridge 211 is irradiated with ultraviolet (UV) by the irradiator provided to the UV radiation unit 226 for a certain period of time, whereby the used reagent cartridge 211 is sterilized.

### (Step S303)

Once the ultraviolet (UV) radiation is completed, the sterilized reagent cartridge 211 is discharged to the outside and discarded.

Hereinafter, the effects of the biological sample processing apparatus 1 according to the present embodiment will be explained.

The biological sample processing apparatus 1 of the present embodiment has the fourth holder 212 for detachably holding the reagent cartridge 211. The reagent cartridge 211 is an example of a liquid supplier. The fourth holder 212 is an example of a holder for detachably holding the liquid supplier. The reagent cartridge 211 has the storage 2111, the piston 2112, and the ejection unit 2113. The storage 2111 stores PBS as a reagent. PBS is an example of a reagent for making an iPS cell. The biological sample processing apparatus 1 has the second pump 213 as a driver for driving the ejector. The second pump 213 is, for example, a syringe pump. The ejection unit 2113 is directly connected to the first filter 207, and the piston 2112 and the ejection unit 2113 push the PBS out from inside the storage 2111 and eject the PBS directly to the first filter 207. The first filter 207 is an example of an ejection destination.

With the above-described configuration, the biological sample processing apparatus 1 of the present embodiment can inject the PBS directly from the reagent cartridge 211 to the first filter 207 by using the reagent cartridge 211, that has both a storage and an ejector, without providing a passage that connects the storage for storing the PBS with the ejection destination. Thus, it is possible to reduce the risk of contamination caused by the attachment of a residue such as a reagent to the inner wall of the passage. Also, it is unnecessary to prepare an extra amount of reagent in anticipation of a liquid feeding loss caused by the attachment of a residue such as a reagent to the inner wall of the passage.

In addition, in a conventional processing apparatus, a multiple-time amount of reagent is stored in a storage such as a reservoir, and a necessary amount of reagent is suctioned from the storage every time cell processing is performed. Since such an apparatus unfailingly suctions a necessary amount of reagent, it is necessary to store, in a reservoir, an amount of reagent that takes an extra amount into consideration. Also, the time during which a reagent is exposed to the external air lengthens while the reagent is stored in a reservoir, likely resulting in an increased risk of contamination. On the other hand, in the present embodiment, an amount of PBS that is used in a single blood cell separation process is prestored in the reagent cartridge 211. Adopting a cartridge-type container in which a reagent is encapsulated in advance makes the suctioning of a reagent unnecessary and can prevent scattering of a reagent caused during suctioning. Also, since the storage is not reused as in the case where a multiple-time amount of reagent is stored, it is possible to reduce the time during which a reagent is exposed to the external air and reduce the risk of microbial contamination and the risk of deterioration caused by long-term use. That is, by adopting single use for which only a necessary amount of reagent consumed in one shot is stored, it is possible to reduce the risk of contamination. In addition, since the size of each component such as a reagent storage can be reduced, it is possible to reduce the distance between the ejection unit of a reagent and the ejection destination and suppress scattering of the reagent caused during ejection of the reagent. A multiple-time amount of reagent may be stored in the reagent cartridge 211.

Furthermore, a conventional processing apparatus has a risk of mixing up reagents if it uses multiple reagent bags and reagent bottles that are commercially available, since reagent bags and reagent bottles have similar outer appearances. On the other hand, the present embodiment provides a mix-up preventer for preventing mix-up of the reagent cartridge 211. As shown in FIGS. 4 and 5, the fourth holder 212 is formed to have a unique shape that allows only its corresponding liquid supplier (the reagent cartridge 211) to be attached thereto, and the reagent cartridge 211 is formed to have a unique shape that allows only its corresponding holder (the fourth holder 212) to be attached thereto, whereby it is possible to prevent a reagent that is not the reagent cartridge 211 from being attached to the fourth holder 212. In this manner, by adopting a unique combination of the shape of the reagent cartridge and the shape of the fitting for each step, it is possible to prevent attaching a reagent to a wrong step, prevent mixing up a reagent or a cell suspension, and reduce the risk of mixing in a reagent or a cell suspension (i.e. risk of contamination). It should be noted that the mix-up preventer may be referred to as a suppressor for suppressing attachment of a liquid supplier to a holder that does not correspond to the liquid supplier.

In addition, a different means may be adopted as a mix-up preventer. For example, an identification code for identifying a type or amount of reagent may be provided on the outer surface of the storage 2111 of the reagent cartridge 211. The identification code is, for example, a bar-code, a symbol, a name of a reagent, etc. In this case, a reader capable of reading an identification code is provided near the fourth holder 212. The reader is, for example, a camera or a bar-code reader and is installed in a position where it can read the identification code of the reagent cartridge 211. The control circuitry 7 acquires, from the reader, a result of reading an identification code and determines whether or not a correct reagent cartridge 211 is attached based on the result of reading an identification code. For example, if a read identification code corresponds to the blood bag 202 or a different reagent bag, the control circuitry 7 determines that a wrong reagent is attached to the fourth holder 212 and warns the operator.

A combination of the fourth holder 212 and a correct reagent to be attached to the fourth holder 212 can be discretionarily set by an operator. For example, a type of correct reagent cartridge attached to the fourth holder 212 and an identification code thereof can be set in advance according to the amount of reagent used.

Also, an operator may determine by him/herself whether or not a correct reagent cartridge 211 is attached. In this case, for example, as a mix-up preventer, an identification marker for identifying a correspondence relationship between the reagent cartridge 211 and the fourth holder 212 is provided to each of the reagent cartridge 211 and the fourth holder 212. The identification marker is, for example, a mark having a specific color or shape. For example, the attaching of marks having the same color or shape to the reagent cartridge 211 and the fourth holder 212 allows an operator to determine by him/herself whether or not a correct reagent is attached to a correct position.

The biological sample processing apparatus 1 has a conveyor for conveying a product generated during processing. In the present embodiment, the conveyor 210 for moving the third collecting container 224 storing PBMCs from the ninth holder 225 is provided as the conveyor, and when PBMCs are stored in the third collecting container 224, the conveyor 210 can automatically convey the third collecting container 224. In a conventional processing apparatus, every time a final product is generated by the processing of a biological sample, it is necessary to process the next biological sample after an operator removes the final product. On the other hand, according to the biological sample processing apparatus 1 of the present embodiment, by moving a final product using the conveyor, it is possible to continuously perform the blood cell separation process without having an operator collect the third collecting container 224 every time a single blood cell separation process is completed. That is, the biological sample processing apparatus 1 can continuously process a biological sample. In this manner, the biological sample processing apparatus 1 can continuously produce PBMCs, and can thus increase the production of PBMCs for each batch. It is also possible to adjust the amount of production by adjusting the conveying speed of the conveyor 210 for performing continuous production according to a target yield.

It is possible to perform a unit operation of performing a single blood cell separation process through a single operation, or an operator may discretionarily set a switch between a unit operation and a continuous operation.

In addition, the blood cell separation mechanism 2 has the UV radiation unit 226 as a waste disposer for disposing of waste generated by the processing of a biological sample. In the UV radiation unit 226, the used reagent cartridge 211 can be irradiated with ultraviolet (UV) and sterilized. Thus, the used reagent can be safely discarded.

### Second Embodiment

A second embodiment will be described. The second embodiment is a modification of the configuration of the first embodiment, as described below. Descriptions of the configuration, operation, and effect that are the same as those of the first embodiment will be omitted. The biological sample processing apparatus 1 of the present embodiment is a factor introduction apparatus for automatically introducing a factor into a peripheral blood mononuclear cell (hereinafter referred to as a “PBMC”) after expansion culture to prepare an iPS cell.

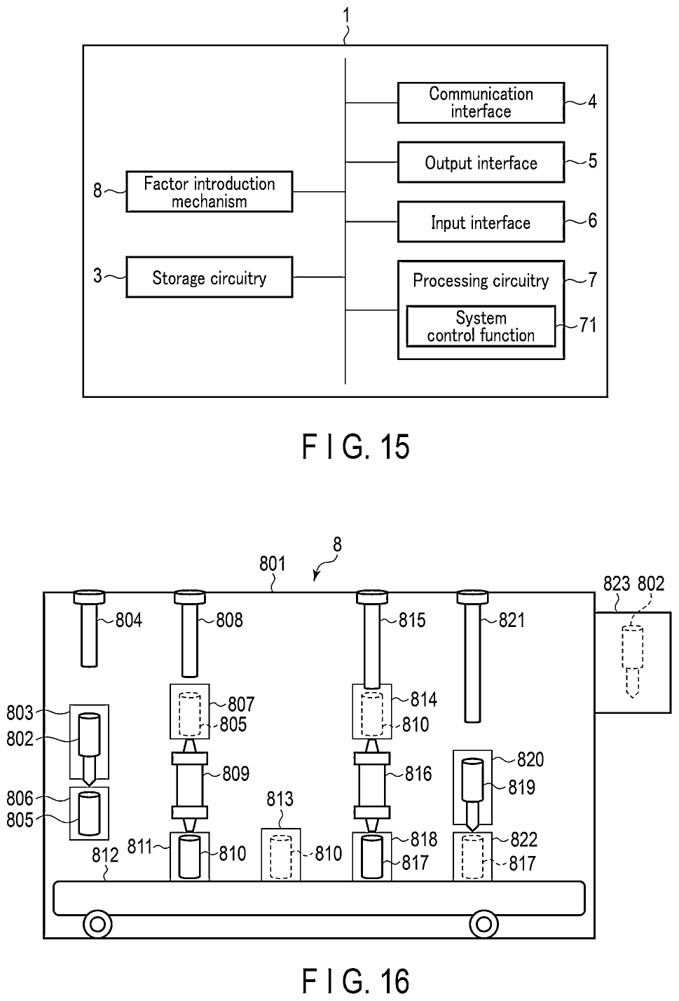

FIG. 15 is a block diagram showing an example of a configuration of the biological sample processing apparatus 1 according to the second embodiment. As shown in FIG. 15, the biological sample processing apparatus 1 of the present embodiment has a factor introduction mechanism 8 instead of the blood cell separation mechanism 2. The factor introduction mechanism 8 introduces a factor into a PBMC after expansion culture. A PBMC is an example of a biological sample.

FIG. 16 is a diagram showing an example of a configuration of the factor introduction mechanism 8 shown in FIG. 15. As shown in FIG. 16, the factor introduction mechanism 8 has a housing 801, a first reagent cartridge 802, a first holder 803, a first pump 804, a PBMC container 805, a second holder 806, a third holder 807, a second pump 808, a first filter 809, a first collecting container 810, a fourth holder 811, a conveyor 812, an infection unit 813, a fifth holder 814, a third pump 815, a second filer 816, a second collecting container 817, a sixth holder 818, a second reagent cartridge 819, a seventh holder 820, a fourth pump 821, an eighth holder 822, and an UV radiation unit 823. The factor introduction mechanism 8 also has a drive mechanism. The drive mechanism actuates each of the above components of the factor introduction mechanism 8 under the control of the control circuitry 7. The drive mechanism is realized, for example, by a robot arm, a pump, a gear, a belt conveyor, etc. For example, the drive mechanism operates each pump or moves each component to any position using a robot arm under the control of the control circuitry 7. For example, a syringe pump is used as the first pump 804, the second pump 808, the third pump 815, and the fourth pump 821. For example, a well plate, a flask, a dish, or a bag are used as the various containers such as the PBMC container 805, the first collecting container 810, and the second collecting container 817.

The housing 801 is an exterior of the factor introduction mechanism 8.

The first reagent cartridge 802 is a cartridge-type container capable of storing a stealth RNA vector (hereinafter referred to as an “SRV”). The first reagent cartridge 802 is an example of a liquid supplier of the present embodiment. A one-time amount of SRV is prestored in the first reagent cartridge 802. The one-time amount is, for example, an amount of SRV used in a single factor introduction process. A multiple-time amount of SRV may be stored in the first reagent cartridge 802. The first reagent cartridge 802 may be referred to as a “factor cartridge”. Since the configuration of the first reagent cartridge 802 is the same as the configuration of the reagent cartridge 211 shown in FIG. 3, description of the configuration of the first reagent cartridge 802 will be omitted. The SRV is an example of a factor.

The first holder 803 is a holding mechanism for holding the first reagent cartridge 802. The first holder 803 is an example of a holder for holding the liquid supplier. The first reagent cartridge 802 is attached to the first holder 803 by an operator. The first holder 803 is arranged directly below the first pump 804. The second holder 806 is arranged directly below the first holder 803.

The first pump 804 is connected to the first reagent cartridge 802 held by the first holder 803. The first pump 804 is, for example, a syringe pump. The first pump 804 extends downward under the control of the control circuitry 7 and ejects the SRV stored in the first reagent cartridge 802 to the PBMC container 805 held by the second holder 806. The first pump 804 is an example of a driver.

The PBMC container 805 is a container capable of storing PBMCs after expansion culture. The PBMCs after expansion culture are supplied from a culturing apparatus that performs a culturing process. For example, the PBMC container 805 has air tightness so as to be able to suppress or prevent exposure of the mixed solution to the external air. Predetermined amounts of PBMCs are prestored in the PBMC container 805.

The second holder 806 is a holding mechanism for holding the PBMC container 805. The PBMC container 805 is attached to the second holder 806 by an operator. The second holder 806 is arranged directly below the first holder 803.

The first reagent cartridge 802 ejects an SRV directly to the PBMC container 805 through the driving done by the first pump 804. The PBMC container 805 is an example of an ejection destination to which a liquid is ejected from the liquid supplier. Once an SRV is ejected to the PBMC container 805, the SRV is introduced into the PBMCs in the PBMC container 805. Thereafter, the PBMC container 805 is conveyed from the second holder 806 to the third holder 807 by a robot arm. The used first reagent cartridge 802 is conveyed from the first holder 803 to the UV radiation unit 823 by a robot arm.

The third holder 807 is a holding mechanism for holding the PBMC container 805 conveyed from the second holder 806. The third holder 807 is arranged between the second pump 808 and the first filter 809.

The second pump 808 is connected to the PBMC container 805 held by the third holder 807. The second pump 808 is, for example, a syringe pump. The second pump 808 ejects the PBMCs stored in the PBMC container 805 to the first filter 809.

The first filter 809 is a filter for performing predetermined filtering on the PBMCs to which the SRV has been introduced. The first filter 809 is arranged directly below the third holder 807.

The first collecting container 810 is a container for collecting the PBMCs having passed through the first filter 809. For example, the first collecting container 810 has air tightness so as to be able to suppress or prevent exposure of the collected liquid to the external air.

The fourth holder 811 is a holding mechanism for holding the first collecting container 810. The fourth holder 811 is positioned directly below the first filter 809. The fourth holder 811 is realized by the conveyor 812. The conveyor 812 is a belt conveyor for conveying the first collecting container 810 and the second collecting container 817. The first collecting container 810 having collected the PBMCs which have passed through the first filter 809 is conveyed to the infection unit 813 by the conveyor 812.

The infection unit 813 is a standby position for infecting the PBMCs in the first collecting container 810 with the SRV. For example, the infection unit 813 is arranged on the conveyor 812. The first collecting container 810 is arranged in the infection unit 813 until a predetermined period of time elapses. When a predetermined period of time elapses, the first collecting container 810 is conveyed to the fifth holder 814 by a robot arm.

The fifth holder 814 is a holding mechanism for holding the first collecting container 810 conveyed from the infection unit 813. The fifth holder 814 is arranged between the third pump 815 and the second filter 816.

The third pump 815 is connected to the first collecting container 810 held by the fifth holder 814. The third pump 815 is, for example, a syringe pump. The third pump 815 ejects the infected PBMCs stored in the first collecting container 810 to the second filter 816.

The second filter 816 is a filter for performing predetermined filtering on the infected PBMCs. The second filter 816 is arranged directly below the fifth holder 814.

The second collecting container 817 is a container for collecting the PBMCs having passed through the second filer 816. For example, the second collecting container 817 has air tightness so as to be able to suppress or prevent exposure of the collected liquid to the external air.

The sixth holder 818 is a holding mechanism for holding the second collecting container 817. The sixth holder 818 is arranged directly below the second filter 816. The second collecting container 817 is conveyed from the sixth holder 818 to the eighth holder 221 by the conveyor 812.

The second reagent cartridge 819 is a cartridge-type container capable of storing a medium such as StemSpan. The second reagent cartridge 819 is an example of a liquid supplier of the present embodiment. A one-time amount of StemSpan is prestored in the second reagent cartridge 819. The one-time amount is, for example, an amount of StemSpan used in a single factor introduction process. A multiple-time amount of StemSpan may be stored in the second reagent cartridge 819. The second reagent cartridge 819 may be referred to as a “collected liquid cartridge”. Since the configuration of the second reagent cartridge 819 is the same as the configuration of the reagent cartridge 211 shown in FIG. 3, description of the configuration of the second reagent cartridge 819 will be omitted.

The seventh holder 820 is a holding mechanism for holding the second reagent cartridge 819. The seventh holder 820 is an example of a holder for holding the liquid supplier. The second reagent cartridge 819 is attached to the seventh holder 820 by an operator. The seventh holder 820 is arranged between the fourth pump 821 and the eighth holder 221.

The fourth pump 821 is connected to the second reagent cartridge 819 held by the seventh holder 820. The fourth pump 821 is, for example, a syringe pump. The fourth pump 821 ejects the StemSpan stored in the second reagent cartridge 819 to the second collecting container 817 held by the eighth holder 221. The fourth pump 821 is an example of a driver.

In the seventh holder 820, the second reagent cartridge 819 is directly connected to the second collecting container 817. The second reagent cartridge 819 ejects StemSpan directly to the second collecting container 817 through the driving done by the fourth pump 821. The second collecting container 817 is an example of an ejection destination to which a liquid is ejected from the liquid supplier. The second reagent cartridge 819 is conveyed from the seventh holder 820 to the UV radiation unit 226 by a robot arm.

The eighth holder 822 is a holding mechanism for holding the second collecting container 817 conveyed from the second holder 806. The eighth holder 822 is realized by the conveyor 812. The second collecting container 817 to which StemSpan has been introduced is conveyed to a slot (not shown) by the conveyor 812. An operator removes the infected PBMCs to which StemSpan has been introduced from the factor introduction mechanism 8 by removing the second collecting container 817 from the slot, and performs the process subsequent to the factor introduction process.

The UV radiation unit 823 stores a used first reagent cartridge 802 and a used second reagent cartridge 819 and irradiates them with ultraviolet (UV). The UV radiation unit 823 is provided with an irradiator for emitting ultraviolet (UV), and the used first reagent cartridge 802 and the used second reagent cartridge 819 are irradiated with ultraviolet for a certain period of time. Once the ultraviolet radiation is completed, the first reagent cartridge 802 and the second reagent cartridge 819 are removed from a discharge port (not shown) to be discharged to the outside and discarded. The UV radiation unit 823 is an example of a waste disposer for disposal of waste generated by the processing of a biological sample.

A small-sized passage or filter provided to the factor introduction mechanism 8 may be configured to be detachable. For example, the first filter 809 and the second filter 816 may be arbitrarily detachable, and may be replaceable with a filter or passage having a desired function. The first filter 809 and the second filer 816 configured to be detachable are examples of a detachable processor used to process a biological sample.

Also, as in the first embodiment, the biological sample processing apparatus 1 has a mix-up preventer for preventing a mix-up of the first reagent cartridge 802 and the second reagent cartridge 819. For example, as the mix-up preventer, the first reagent cartridge 802, the first holder 803, the second reagent cartridge 819, and the seventh holder 820 are formed to allow only a reagent cartridge corresponding thereto to be attached thereto.

For example, the first reagent cartridge 802 and the first holder 803 are formed to have the shape shown in the example in FIG. 4, and the second reagent cartridge 819 and the seventh holder 820 are formed to have the shape shown in the example in FIG. 5. In this case, the first reagent cartridge 802 cannot be attached to the seventh holder 820 and can only be attached to the first holder 803. Also, the second reagent cartridge 819 cannot be attached to the first holder 803 and can only be attached to the seventh holder 820.

Next, an operation of the biological sample processing apparatus 1 of the present embodiment will be described.

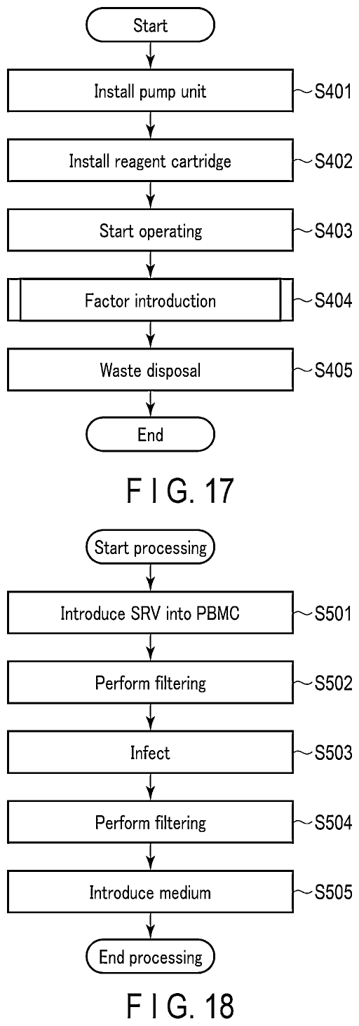

FIG. 17 is a flowchart showing an example of a factor introduction process procedure performed by an operator using the biological sample processing apparatus 1 of the present embodiment. The factor introduction process is a process of introducing a factor such as a SRV or StemSpan into a PBMC which has undergone a culturing process in the preparation of an iPS cell. FIG. 18 is a flowchart showing an example of a procedure of factor introduction performed in step S404 in FIG. 17. The procedure of each step described below is a mere example, and each step can be altered as appropriate to the extent possible. Omission, replacement, or addition of a step in the process procedure described below can be made as appropriate according to the manner in which the present embodiment is realized.

### (Factor Introduction Process)

### (Step S401)

First, an operator installs a pump unit including the first pump 804, the second pump 808, the third pump 815, and the fourth pump 821 in a predetermined position inside the housing 801 of the factor introduction mechanism 8. The pump unit may be referred to as a “nozzle unit”.

### (Step S402)

Next, the operator installs the first reagent cartridge 802 in which a SRV is prestored in the first holder 803, and installs the second reagent cartridge 819 in which StemSpan is prestored in the seventh holder 820. At this time, the operator can install the first reagent cartridge 802 and the second reagent cartridge 819 in a correct position without mixing them up since the first reagent cartridge 802, the first holder 803, the second reagent cartridge 819, and the seventh holder 820 are provided with the aforementioned mix-up prevention function.

### (Step S403)

Next, the operator performs an operation to start operating the biological sample processing apparatus 1 through the input interface 6.

### (Step S404)

When the biological sample processing apparatus 1 starts to operate, the biological sample processing apparatus 1 performs the factor introduction under the control of the control circuitry 7. In the factor introduction, an operation of introducing a factor into a PBMC is automatically performed by the factor introduction mechanism 8 under the control of the control circuitry 7.

Herein, the factor introduction performed by the biological sample processing apparatus 1 will be explained in detail.

FIG. 18 is a flowchart showing an example of a procedure of the factor introduction performed by the biological sample processing apparatus 1. Each processing described below is performed through the driving done by a drive mechanism such as a robot arm or the conveyor 812 under the control of the control circuitry 7, a description of which is omitted.

### (Factor Introduction)

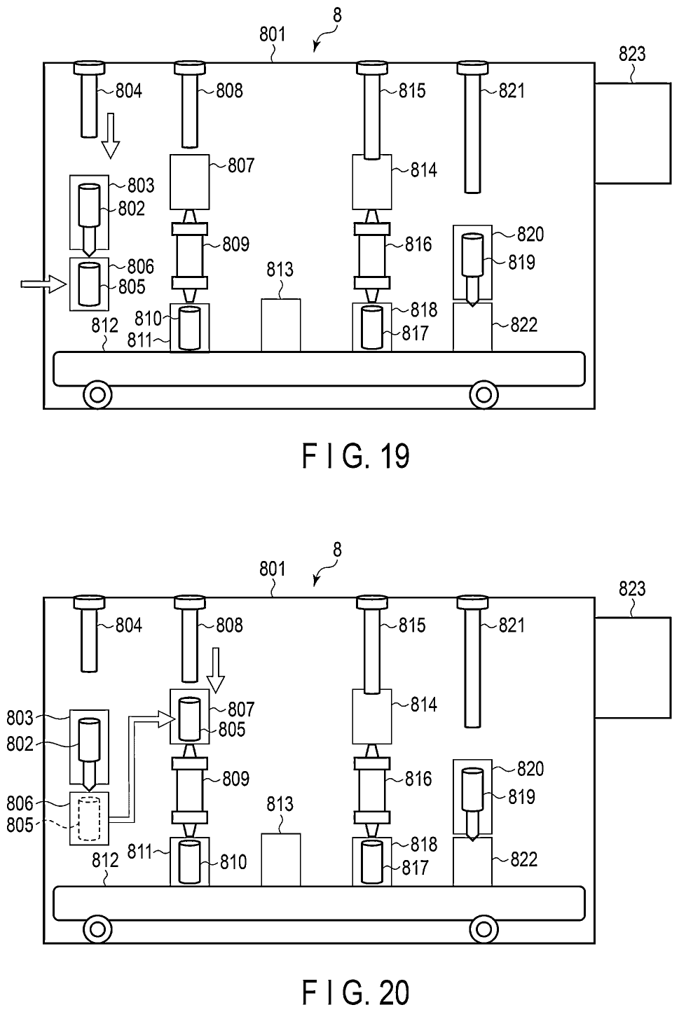

FIG. 19 is a diagram showing an internal state of the factor introduction mechanism 8 when the factor introduction is started. As shown in FIG. 19, when the factor introduction is to be commenced, the first reagent cartridge 802 is attached to the first holder 803, and the second reagent cartridge 819 is attached to the seventh holder 820. The PBMC container 805 storing the PBMCs after the culturing process is installed in the second holder 806. The first collecting container 810 is installed in the fourth holder 811, and the second collecting container 817 is installed in the sixth holder 818.

### (Step S501)

As shown in FIG. 19, when the factor introduction is commenced, an SRV stored in the first reagent cartridge 802 is ejected to the PBMC container 805 through the driving done by the first pump 206, and the SRV is introduced into the PBMCs in the PBMC container 805.

### (Step S502)

Next, the PBMC container 805 moves from the second holder 806 to the third holder 807, as shown in FIG. 20. Then, through the driving done by the second pump 808, the PBMCs with a SRV that are stored in the PBMC container 805 are ejected to the first filter 809 and subjected to filtering by the first filter 809. The PBMCs having passed through the first filter 809 are collected in the first collecting container 810 installed in the fourth holder 811.

### (Step S503)

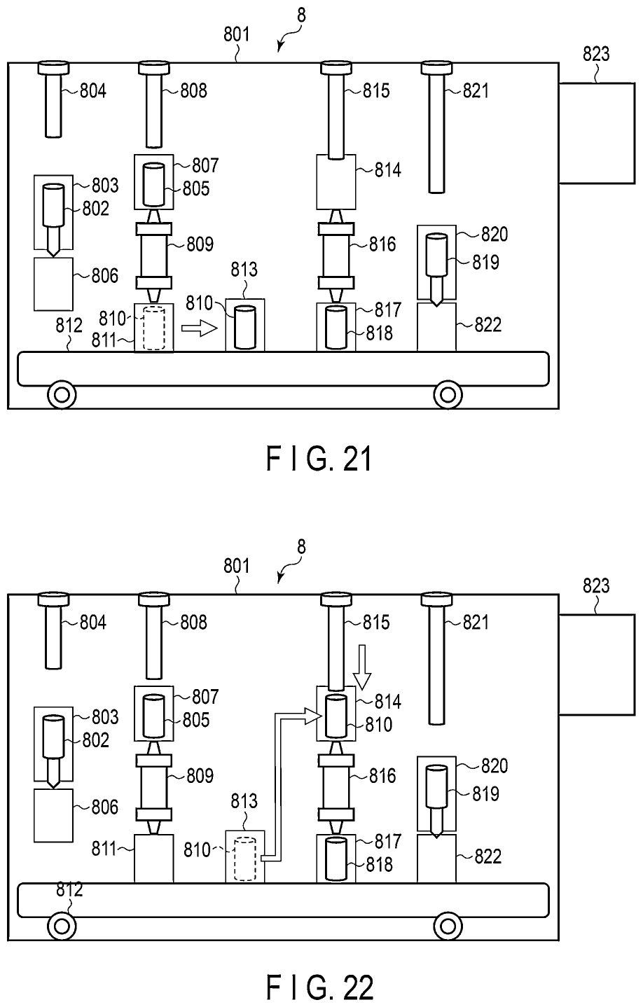

Next, the first collecting container 810 is conveyed to the infection unit 813, as shown in FIG. 21. The first collecting container 810 puts itself on stand-by in the infection unit 813 until a predetermined period of time elapses and the PBMCs are infected with a SRV.

### (Step S504)

When a predetermined period of time elapses, the first collecting container 810 moves from the infection unit 813 to the fifth holder 814, as shown in FIG. 22. Then, through the driving done by the third pump 815, the infected PBMCs stored in the first collecting container 810 is ejected to the second filter 816 and subjected to filtering by the second filter 816. The PBMCs having passed through the second filter 816 are collected in the second collecting container 817 installed in the sixth holder 818.

### (Step S505)

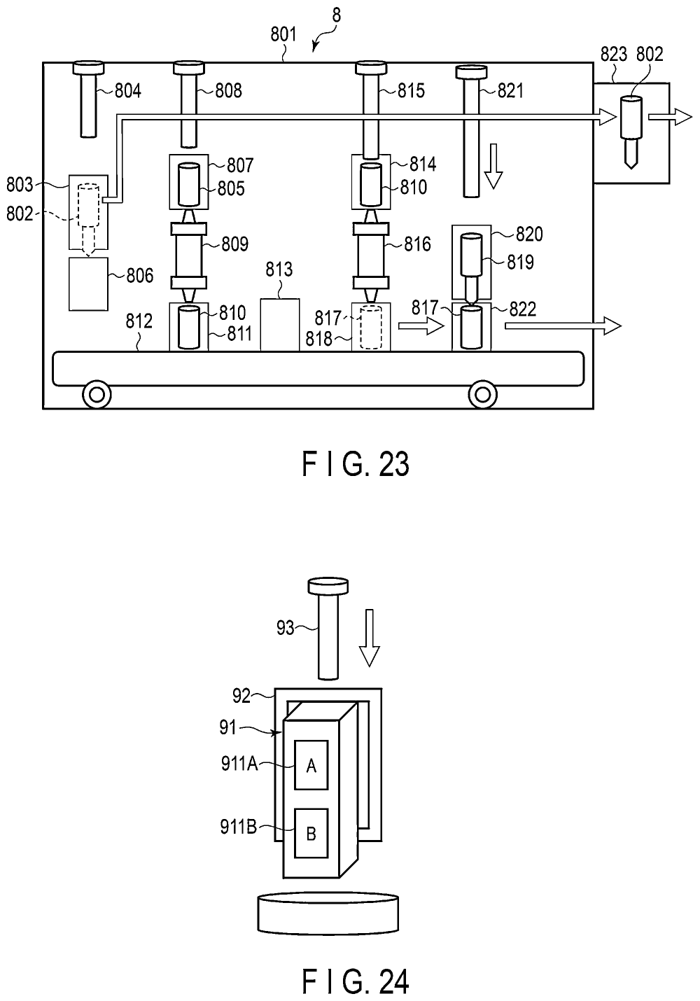

Next, the second collecting container 817 moves from the sixth holder 818 to the eighth holder 822, as shown in FIG. 23. Then, through the driving done by the fourth pump 821, the StemSpan stored in the second reagent cartridge 819 is ejected to the second collecting container 817 as a medium and the StemSpan is introduced into the infected PBMCs.

In this manner, in the factor introduction performed in step S404, a SRV is introduced into the PBMCs necessary to process iPS cells through a series of steps S501 through S505, and StemSpan is introduced into the infected PBMCs.

### (Step S405)

Once the factor introduction performed in step S404 is completed, the operator conveys the second collecting container 817 from the eighth holder 822 to the slot, as shown in FIG. 23, and completes the factor introduction process. Simultaneously, the operator performs waste disposal using the biological sample processing apparatus 1. In the waste disposal, the used first reagent cartridge 802 having moved to the UV radiation unit 823 is irradiated with ultraviolet (UV) for a certain period of time, as in steps S301 to S303 shown in FIG. 8. Once the ultraviolet (UV) radiation is completed, the sterilized first reagent cartridge 802 is discharged to the outside and discarded. The same type of waste disposal as that performed on the first reagent cartridge 802 may be performed on the used second reagent cartridge 819.

Hereinafter, the effects of the biological sample processing apparatus 1 according to the present embodiment will be explained.

The biological sample processing apparatus 1 of the present embodiment has the first holder 803 for detachably holding the first reagent cartridge 802 and the seventh holder 820 for detachably holding the second reagent cartridge 819. The first reagent cartridge 802 and the second reagent cartridge 819 are examples of a liquid supplier. The first holder 803 and the seventh holder 820 are examples of a holder for detachably holding the liquid supplier.

The first reagent cartridge 802 has a storage for storing a SRV as a reagent and an ejector for pushing the SRV out from inside the storage and ejecting the SRV directly to the PBMC container 805. A SRV is an example of a reagent for making an iPS cell. The biological sample processing apparatus 1 also has the first pump 804 as a driver for driving the ejector. The first pump 804 is, for example, a syringe pump. The first reagent cartridge 802 is directly connected to the PBMC container 805 and pushes the SRV out from inside the storage to eject the SRV directly to the PBMC container 805. The PBMC container 805 as a storing container is an example of an ejection destination.

The second reagent cartridge 819 has a storage for storing StemSpan as a reagent and an ejector for pushing the StemSpan out from inside the storage and directly ejecting the StemSpan to the second collecting container 817. StemSpan is an example of a reagent for making an iPS cell. The biological sample processing apparatus 1 also has the fourth pump 821 as a driver for driving the ejector. The fourth pump 821 is, for example, a syringe pump. The second reagent cartridge 819 is directly connected to the second collecting container 817 and pushes the StemSpan out from inside the storage to eject the StemSpan directly to the second collecting container 817. The second collecting container 817 as a storing container is an example of an ejection destination.

With the above-described configuration, the biological sample processing apparatus 1 of the present embodiment can introduce a reagent directly into an ejection destination by using the first reagent cartridge 802 and the second reagent cartridge 819 that have both a storage and an ejector without providing a passage that connects the storage with the ejection destination. Thus, the same effects as those achieved by the first embodiment can be achieved. For example, it is possible to reduce the risk of contamination and a liquid feeding loss caused by the attachment of a residue such as a reagent to the inner wall of the passage, eliminating the need to prepare an extra amount of reagent in anticipation of a liquid feeding loss.

Also, as in the first embodiment, since single use, for which only a necessary amount consumed in one shot is stored, is adopted for the first reagent cartridge 802 and the second reagent cartridge 819, it is possible to reduce the risk of contamination.

The present embodiment also provides a mix-up preventer for preventing mix-up of the first reagent cartridge 802 and the second reagent cartridge 819. Thus, as in the first embodiment, it is possible to prevent attaching a reagent to a wrong step, prevent the mixing up a reagent or a cell suspension, and reduce the risk of mixing in a reagent or a cell suspension (i.e. risk of contamination). For example, the first reagent cartridge 802 and the first holder 803 are formed to have a unique shape that allows them to be attached only to each other, and the second reagent cartridge 819 and the seventh holder 820 are formed to have a unique shape that allows them to be attached only to each other, and whereby a mix-up of the first reagent cartridge 802 and the second reagent cartridge 819 can be prevented. As in the first embodiment, another means of mix-up preventer may be used.

Since the present embodiment also provides the conveyor 812 as a conveyor for conveying a product generated during the processing step, it is possible to continuously produce PBMCs into which a SRV and StemSpan have been introduced, as in the first embodiment. It is also possible to perform a unit operation consisting of the performing a single factor introduction process through a single operation. Alternatively, an operator may discretionarily set a switch between a unit operation and a continuous operation.

In the present embodiment as well, the factor introduction mechanism 8 has the UV radiation unit 823 as a waste disposer for disposing of waste generated by the processing of a biological sample; thus, a used reagent can be safely discarded, as in the first embodiment.

### (First Modification)

A reagent cartridge that can separately store two kinds of reagents that are mixed together immediately before the processing step may be used. FIG. 24 is a diagram showing an example of a configuration of a reagent cartridge 91 according to a first modification. The reagent cartridge 91 is held by a holder 92 and connected to a pump 93.

The reagent cartridge 91 has a first storage 911A and a second storage 911B. Although not shown, the reagent cartridge 91 has a piston and an ejection unit as an ejector. The first storage 911A and the second storage 911B are reagent containers for respectively storing different kinds of reagents. The first storage 911A stores liquid A as a reagent, and the second storage 911B stores liquid B as a reagent. For example, two kinds of reagents that need to be mixed together immediately before the step of processing a biological sample are stored in the first storage 911A and the second storage 911B. Through the driving done by the pump 93, the reagents are ejected from the first storage 911A and the second storage 911B, respectively, to ejection destinations such as a collecting container. In the container as an ejection destination, the reagents ejected from the first storage 911A and the second storage 911B, respectively, are mixed together. In this manner, by using the reagent cartridge 91 that can separately store two kinds of mixed-together reagents immediately before the processing step and simultaneously eject these reagents, it is possible to mix the reagents together without preparing separate reagent containers for respective reagents or providing a passage between the reagent containers. Since it is unnecessary to provide a passage between reagent containers, it is possible to prevent the reagents from remaining in a passage. A reagent cartridge that can separately store three or more kinds of reagents may be used.

### (Second Modification)

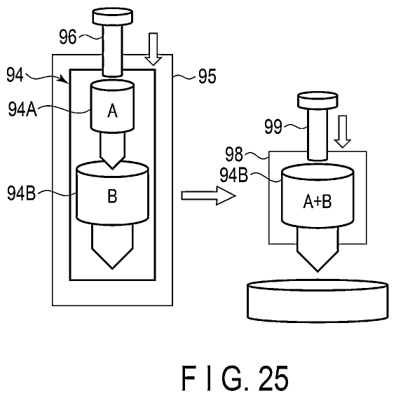

A reagent cartridge that mixes two kinds of reagents in a cartridge may be used. FIG. 25 is a diagram showing an example of a configuration of a reagent cartridge 94 according to a second modification. The reagent cartridge 94 is held by a first holder 95 and connected to a first pump 96.

The reagent cartridge 94 has a first cartridge 94A and a second cartridge 94B. The first cartridge 94A and the second cartridge 94B are detachably connected to each other inside the reagent cartridge 94. The first cartridge 94A and the second cartridge 94B are reagent containers for respectively storing different kinds of reagents. The first cartridge 94A stores liquid A as a reagent, and the second cartridge 94B stores liquid B as a reagent. For example, two kinds of reagents that need to be mixed together when the step of processing a biological sample is commenced are stored in the first cartridge 94A and the second cartridge 94B.

Each of the first cartridge 94A and the second cartridge 94B has a storage for storing a reagent and has a piston and an ejection unit as an ejector for ejecting the reagent from the storage. Inside the reagent cartridge 91, the ejection unit of the first cartridge 94A is connected to the storage of the second cartridge 94B. The first pump 96 is connected to the piston of the first cartridge 94A.

In the case of mixing two reagents together immediately before the processing step, first, the reagent A stored in the first cartridge 94A is injected from the ejection unit of the first cartridge 94A into the storage of the second cartridge 94B through the driving done by the first pump 96. Inside the storage of the second cartridge 94B, the liquid A and the liquid B are mixed together.

When the liquid A and the liquid B are mixed together, the second cartridge 94B is removed from the reagent cartridge 94, and then moved to the second holder 98 by the drive mechanism such as a robot arm. In the second holder 98, the second cartridge 94B is connected to the second pump 99. Also, through the driving done by the second pump 99, the mixture of the liquid A and the liquid B stored in the second cartridge 94B is ejected to an ejection destination such as a collecting container. In this manner, by using the reagent cartridge 94 that can mix, inside the cartridge, two kinds of reagents that are to be mixed together immediately before the processing step, it is possible to mix the reagents together without preparing separate reagent containers for respective reagents or providing a passage between the reagent containers. Since it is unnecessary to provide a passage between reagent containers, it is possible to prevent the reagents from remaining in a passage. A reagent cartridge that can separately store three or more kinds of reagents may be used.

### Application Example

In the examples shown in the above embodiments and modifications, the biological sample processing apparatus is an apparatus that automatically performs a blood cell separation process or a factor introduction process for making an iPS cell; however, the biological sample processing apparatus is not limited thereto. For example, the biological sample processing apparatus may be a culturing apparatus that automatically performs cell culturing, a dispensing apparatus that automatically dispenses a liquid, or a cell processing apparatus that automatically performs various kinds of processing on various kinds of cells. That is, a biological sample processed by the biological sample processing apparatus may be blood, a blood suspension, or a PBMC, or it may be a culture liquid. Also, a liquid used for processing a biological sample may be a medium or a factor used for cell culturing or a reagent or a different liquid used for various kinds of cell processing.

In any of the above cases, by using a cartridge-type liquid supplier that can store a liquid used for processing a biological sample and eject the liquid directly to an ejection destination, it is possible to supply a liquid to an ejection destination without causing the liquid to pass through a passage and reduce the risk of contamination caused by a biological sample or liquid. Also, in any of the cases described above, by applying a mix-up preventer to the liquid supplier or the holder of the liquid supplier, it is possible to prevent mix-ups of multiple kinds of liquids used for the processing.

The above-described cartridge-type liquid supplier may be applied not only to a commercially available reagent bag storing PBS, a SRV, and StemSpan but also to a container storing an intermediate product generated during a processing step. For example, the mixing container 203, the first collecting container 214, and the second collecting container 219 according to the first embodiment or the PBMC container 805 and the first collecting container 810 according to the second embodiment may be used as the cartridge-type liquid supplier.

Also, in the examples shown in the above embodiments, a piston structure in which to drive using a pump is provided as the ejector provided to the liquid supplier; however, the ejector is not limited thereto. For example, positive pressure may be created inside the storage so that a liquid is automatically ejected to an ejection destination connected thereto. Also, an operator may manually drive the piston. In this case, a driver (drive unit) such as a pump becomes unnecessary. Also, the storage may be formed in a structure that allows an operator to squash the storage from its two side surfaces with his or her hands. In this case, the ejection port and the squashing structure provided to the storage correspond to the ejector, making a driver (drive unit) such as a pump unnecessary.

According to at least one embodiment described above, it is possible to reduce the risk of contamination occurring during processing of a biological sample.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A biological sample processing apparatus comprising a holder for detachably holding a liquid supplier including a storage for storing a liquid used for processing a biological sample and an ejector for pushing the liquid out from inside the storage to eject the liquid directly to an ejection destination.

2. The biological sample processing apparatus according to claim 1, wherein the storage is configured to store the liquid in an amount corresponding to one-time use.

3. The biological sample processing apparatus according to claim 1, wherein the storage is configured to store the liquid in an amount corresponding to multiple-time use.

4. The biological sample processing apparatus according to claim 1, further comprising a driver configured to drive the ejector to eject the liquid.

5. The biological sample processing apparatus according to claim 1, further comprising a conveyor for conveying a product generated during the processing.

6. The biological sample processing apparatus according to claim 5, configured to continuously process the biological sample.

7. The biological sample processing apparatus according to claim 1, further comprising a mix-up preventer for preventing mix-up of the liquid supplier.

8. The biological sample processing apparatus according to claim 7, wherein the mix-up preventer is provided at the storage and includes an identification code for identifying the liquid and a reader for reading the identification code.

9. The biological sample processing apparatus according to claim 8, wherein the mix-up preventer further comprises a controller configured to determine whether or not a correct liquid supplier is attached to the holder based on a result of reading the identification code.

10. The biological sample processing apparatus according to claim 7, wherein the mix-up preventer comprises the holder to which only a corresponding liquid supplier is attachable.

11. The biological sample processing apparatus according to claim 7, wherein the mix-up preventer is provided at each of the liquid supplier and the holder and includes an identification marker for identifying a correspondence relationship between the liquid supplier and the holder.

12. The biological sample processing apparatus according to claim 1, further comprising a detachable processor used for the processing of the biological sample.

13. The biological sample processing apparatus according to claim 1, further comprising a waste disposer for disposing of waste generated by the processing.

14. The biological sample processing apparatus according to claim 4, wherein the driver is a syringe pump.

15. The biological sample processing apparatus according to claim 14, wherein the ejector comprises a piston configured to be driven by the syringe pump for movement.

16. The biological sample processing apparatus according to claim 1, wherein
the biological sample is blood for making an induced pluripotent stem cell,
the processing includes separation of a blood cell in the blood, and
the liquid is phosphate buffered saline (PBS).

17. The biological sample processing apparatus according to claim 1, wherein
the biological sample is a peripheral blood mononuclear cell (PBMC) for making an induced pluripotent stem cell,
the processing includes the introduction of a factor into the peripheral blood mononuclear cell, and
the liquid is the factor.

18. The biological sample processing apparatus according to claim 1, wherein the liquid is an intermediate product generated while an induced pluripotent stem cell is being made.

19. The biological sample processing apparatus according to claim 1, wherein the ejection destination is a filter or a storing container.
